(19) United States
(12) Patent Application Publication
(10) Pub. No.: US 2015/0342782 A1
(43) Pub. Date: Dec. 3, 2015

(54) Treatment Systems for Vitreous Floaters

(71) Applicant: Strathspey Crown Holdings, LLC, Newport Beach, CA (US)

(72) Inventors: David Haydn Mordaunt, Los Gatos, CA (US); Andrew Brian Merkur, Vancouver (CA); David Tat-Chi Lin, Vancouver (CA)

(73) Assignee: Strathspey Crown Holdings, LLC, Newport Beach, CA (US)

(21) Appl. No.: 14/291,843

(22) Filed: May 30, 2014

(51) Int. Cl. A61F 9/008

(52) U.S. Cl. CPC A61F 9/00802; A61F 2009/00874; A61F 2009/00882

(57) ABSTRACT

In accordance with the present invention, a system and method are provided for using a computer-controlled, laser system to perform a partial vitrectomy of the vitreous humor in an eye. Operationally, an optical channel is first defined through the vitreous humor. Vitreous and suspended deposits (floaters) in the optical channel are then ablated and in some cases removed (e.g. aspirated) from the optical channel. In some instances, a clear liquid can be introduced into the optical channel to replace the ablated matter, and to thereby establish unhindered transparency in the optical channel.

### FIELD OF THE INVENTION

In general, the present invention pertains to systems and methods for ophthalmic laser surgeries. More particularly, the present invention pertains to systems and methods for using pulsed laser beams to remove so-called “floaters” (i.e. deposits, visual obstructions) from the vitreous humor of an eye. The present invention is particularly, but not exclusively, useful as a system and method for performing a partial vitrectomy of a defined optical channel in the vitreous humor.

### BACKGROUND OF THE INVENTION

Floaters are deposits (i.e. optical obstructions) that form in the vitreous humor of an eye for any of several different reasons. Typically, floaters result from degenerative changes in the gel-like vitreous of the vitreous humor. The consequence of floaters is that they absorb, reflect or refract light as it passes through the vitreous humor. These changes then manifest themselves as so-called “blind spots” and relative scotomas for the individual. At the very least, floaters are an annoyance. They can, however, become significantly disruptive to an individual's vision and quality of life.

From an optical perspective, image perception by an eye relies on light that enters the vitreous humor from the front of the eye, through the pupil and crystalline lens. This light is then focused by the crystalline lens, and passes through the vitreous humor to be incident on the retina of the eye. Importantly, the focused light needs to be incident on the macula and retinal tissue immediately surrounding the macula. As a practical matter, all light which contributes to the imaging capability of the eye will pass through the vitreous humor, and be confined within what is hereby defined as an optical channel.

For purposes of the present invention, the optical channel will be generally cylindrical-shaped. It will have a cross-section diameter of greater than 5 mm. And, it will extend from the posterior surface of the crystalline lens to the retina with a safety margin respectively between the posterior surface of the crystalline lens and the retina of around fifty microns.

With the above in mind, it is an object of the present invention to provide a system and method for performing a partial vitrectomy within a defined optical channel in the vitreous humor. Another object of the present invention is to provide a system and method for removing floaters (i.e. visual obstructions) from the vitreous of an eye with a minimal vitrectomy. Still another object of the present invention is to provide a system and method for removing floaters from the vitreous humor of an eye that is simple to use, is easy to implement, and is commercially cost effective.

### SUMMARY OF THE INVENTION

The purpose of the present invention is to provide a method, a system, and a computer medium which will effectively remove a deposit, such as a floater or a less mobile vitreous opacity, from an eye. In particular, the objective in each case is to establish a transparent optical channel that is free of deposits, and which extends through the vitreous humor from the posterior surface of the crystalline lens to the retina. To do this, a partial vitrectomy in the optical channel is performed using a computer-programmed laser unit which ablates (i.e. liquefies and/or vaporizes) the targeted vitreous. In some instances, the liquefied/vaporized vitreous can then be aspirated and replaced with a clear, natural aqueous fluid, such as a salt solution.

As envisioned for the present invention, the optical channel is defined relative to a previously identified axis. For this purpose, the identified axis can be a visual axis, an optical axis, a central axis, or some other axis well known in the pertinent art which is anatomically oriented on the eye. Based on the selected axis, the optical channel is established to extend through the vitreous humor. It will typically extend from a distance more than fifty microns (50 μm) from the posterior surface of the eye's crystalline lens to a distance more than fifty microns (50 μm) anterior to the retina of the eye. Further, the optical channel is substantially cylindrical shaped, and it extends radially outward to a radial distance r from the axis. Typically, r will be greater than about 2.5 mm. In some cases, the optical channel can be formed with a slightly increasing or decreasing taper as it extends in a posterior direction through the vitreous humor. Importantly, the optical channel will be defined to overlie (i.e. cover) the macula of the retina of the eye.

Structurally, a system for clearing deposits from an optical channel in an eye includes a laser unit and a control unit for moving the focal point of a laser beam through the optical channel. In this combination, an imaging unit is provided for creating an anatomical profile of the vitreous humor of the eye. In particular, this anatomical profile will show the relationship of the vitreous humor with the crystalline lens and with the retina of the eye. Also included here is a programming unit that uses parameters obtained from the anatomical profile to define a laser pathway through the vitreous of the vitreous humor in the optical channel. A computer, which is connected in combination with both the imaging unit and the programming unit, obtains information respectively from these units regarding the anatomical profile and the pathway. The computer then uses this information for collective use in creating a control input to the laser unit.

For the present invention, the laser unit generates a laser beam, and it moves the focal point of the laser beam along the pathway through the vitreous humor in accordance with the control input. The purpose here is to ablate (i.e. liquefy/vaporize) vitreous in the optical channel, as well as any deposits such as floaters that may also be located (suspended) in the optical channel. In some cases, an aspirator can then be used to remove the liquefied vitreous and deposits such as floaters from the optical channel. Simultaneously, an introducer can be used to replace the liquefied vitreous humor, and the deposits, that have been removed from the eye. Alternatively, the gases resulting from ablation (i.e. air bubbles of N₂, CO₂, O₂) may be beneficially left in the eye.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of the operative components of the present invention;

FIG. 2 is a cross-section view of an eye showing an optical channel, anatomically defined and oriented in the eye for purposes of the present invention; and

FIG. 3 shows a cross-section view of the eye as seen in FIG. 2, after floaters have been removed from the optical channel.

### DESCRIPTION OF THE PREFERRED EMBODIMENTS

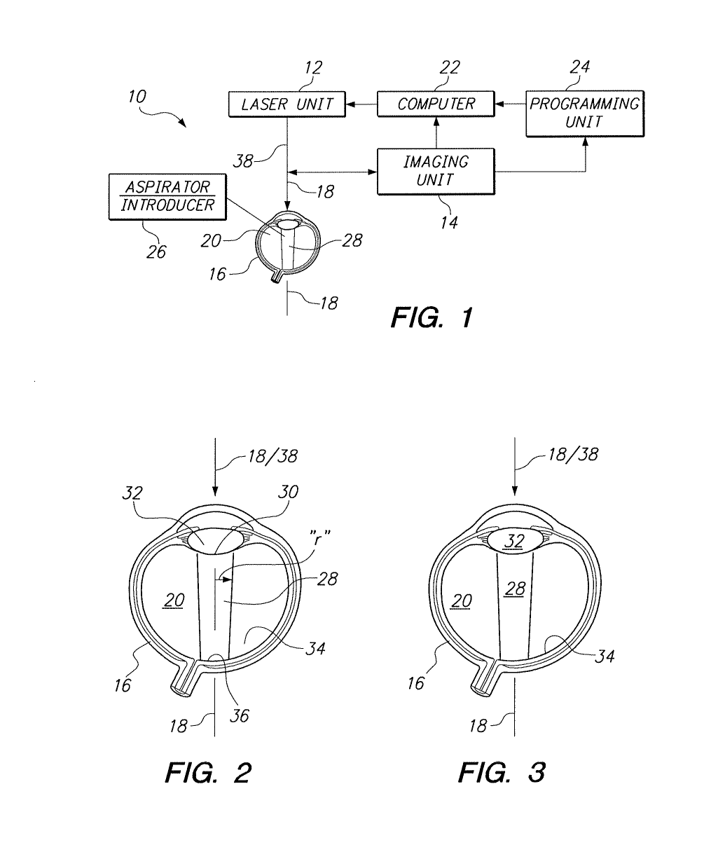

Referring initially to FIG. 1 a system in accordance with the present invention is shown and is generally designated 10. As shown, the system 10 includes a laser unit 12, and an imaging unit 14, that are each respectively positioned for optical interaction with an eye 16. More specifically, the laser unit 12 and the imaging unit 14 are positioned to direct their respective light beams along an axis 18.

For the present invention, the axis 18 is defined relative to selected anatomical features of the eye 16, and it will normally be a reference base that is well known in the pertinent art, such as a visual axis, a central axis or an optical axis. The laser unit 12 may also be of a type that is well known in the pertinent art and is capable of generating a pulsed femtosecond laser beam 38 (i.e. a beam having a sequence of laser pulses with ultra-short pulse durations [e.g. less than approximately 500 fs]). In particular, a laser beam 38 capable of passing through tissue to a subsurface focal point to perform Laser Induced Optical Breakdown (LIOB) of vitreous in the vitreous humor 20 of the eye 16 is to be used. In addition, the laser unit 12 can include a beam steering component for moving the focal spot of the laser beam 38 along a selected path to photoablate target tissue via LIOB. For example, the beam steering component can include a pair of mirrors (not shown) mounted on respective tip-tilt actuators to steer the laser beam 38 in respective, orthogonal directions. Further, the imaging unit 14 is typically of a type that is capable of creating a three-dimensional image of anatomical features in the eye 16, such as an Optical Coherence Tomography (OCT) imaging system, or any other suitable imaging device that is well known in the pertinent art such as a Scheimpflug device, a confocal imaging device, an optical range-finding device, an ultrasound device or a two-photon imaging device.

FIG. 1 also shows that the system 10 includes a computer 22 which is electronically connected with the imaging unit 14 and with the laser unit 12. A programming unit 24 which is electronically connected between the imaging unit 14 and the computer 22 is also included. In detail, the computer 22 receives input from both the imaging unit 14 and the programming unit 24, and it uses this input to control the laser unit 12 in accordance with a predetermined protocol. It is also seen in FIG. 1 that the system 10 can include an optional stand-alone aspirator/introducer unit 26 that can be operated in concert with the other electronically-interconnected components of the system 10.

Referring now to FIG. 2, an optical channel 28 is shown extending through the vitreous humor 20. As indicated above, the optical channel 28 is defined in its relationship with the axis 18. In detail, the optical channel 28 is substantially cylindrical shaped, and it is characterized by a radius r that extends radially outward from the axis 18. Typically, r will be greater than about 2.5 mm. With these dimensional characteristics, the optical channel 28 is established to extend through the vitreous humor 20. In this case, the optical channel 28 will extend from a distance that will be more than about fifty microns (50 μm) behind the posterior surface 30 of the crystalline lens 32 of the eye 16, to a distance that will be more than about fifty microns (50 μm) anterior to the retina 34 of the eye 16. An important consideration for placement of the optical channel 28 in the vitreous humor 20 of the eye 16 is the requirement that the optical channel 28 needs to cover, or overlie, the macula 36 of the retina 34.

For an operation of the system 10 of the present invention, the imaging unit 14 is first used to create an anatomical profile of the vitreous humor 20 of the eye 16. Specifically, this anatomical profile identifies the dimensional relationship between the crystalline lens 32 and the retina 34 of the eye 16. The programming unit 24, which is electronically connected to the imaging unit 14, is used to locate the optical channel 28 in the vitreous humor 20. Once the optical channel 28 has been defined and located in the eye 16, the programming unit 24 defines a pathway (not shown) through the portion of the vitreous humor 20 that is in the optical channel 28. Importantly, the pathway is detailed according to parameters obtained from the anatomical profile that has been created by the imaging unit 14.

As noted above, the computer 22 is connected to the imaging unit 14, and to the programming unit 24. With these connections, the computer 22 obtains the necessary information regarding the anatomical profile and the pathway that is required to create a control input for the laser unit 12. Operationally, this control input is then used by the laser unit 12 to generate the laser beam 38. The computer 22 also uses this control input for moving a focal point of the laser beam 38 along the pathway in the vitreous humor 20. Specifically, all of this is done in accordance with the control input to operate the laser unit 12 for ablating (i.e. liquefying/vaporizing) vitreous in the vitreous humor 20, along with any deposits such as floaters that may be located within the optical channel 28.

As vitreous and deposits such as floaters within the optical channel 28 are being ablated, in some cases, the optional aspirator/introducer unit 26 is activated to remove ablated material from the optical channel 28. Simultaneously, the optional aspirator/introducer unit 26 can replace the liquefied material with a clear aqueous fluid to establish transparency in the optical channel 28. The consequence of this is shown in FIG. 3 where it will be seen that the optical channel 28 is effectively clear of obstructions or deposits (i.e. floaters and scotomas) to provide a transparent optical channel 28 for improved vision.

While the particular Treatment Systems for Vitreous Floaters as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for removing deposits from an optical channel to establish transparency in the channel, wherein the optical channel is defined to extend through an otherwise clear gel-like substance in the channel, the method comprising the steps of:
identifying an axis, wherein the axis extends through the gel-like substance;
defining an optical channel through the gel-like substance, wherein the optical channel is substantially cylindrical shaped and extends radially outward to a distance r from the axis; and
ablating the gel-like substance in the optical channel, and deposits suspended therein.

2. A method as recited in claim 1 further comprising the steps of:
aspirating the ablated gel-like substance to remove the deposits therewith from the optical channel; and
introducing a fluid into the optical channel to replace the aspirated gel-like substance and the deposits removed therefrom during the aspirating step to establish a transparent optical channel and wherein the ablating step is accomplished using a laser beam to photoalter the gel-like substance and the deposits in the optical channel.

3. A method as recited in claim 1 wherein the gel-like substance is the vitreous humor of an eye and the optical channel extends between the posterior surface of the lens of the eye and the retina of the eye.

4. A method as recited in claim 3 wherein the ablating step is accomplished in the optical channel at a distance beyond fifty microns (50 μm) from the posterior surface of the lens and at a distance beyond fifty microns (50 μm) from the retina of the eye.

5. A method as recited in claim 3 wherein the axis is selected from the group consisting of an optical axis, a visual axis, and a central axis.

6. A method as recited in claim 3 wherein the optical channel is located to overlie the macula of the eye.

7. A method as recited in claim 3 wherein the deposits include at least one floater.

8. A method as recited in claim 1 wherein the distance r is greater than 2.5 mm.

9. A method as recited in claim 1 wherein the deposits include at least one effect of vitreous opacity.

10. A system for removing floaters from an optical channel in an eye to establish transparency in the channel, wherein the optical channel is defined to extend through the vitreous humor of the eye, and the system comprises:
an imaging unit for creating an anatomical profile of the vitreous humor of the eye in its relationship with the crystalline lens and the retina of the eye;
a programming unit connected to the imaging unit for defining a pathway through the vitreous in the optical channel, wherein the pathway is detailed according to parameters obtained from the anatomical profile;
a computer connected to the imaging unit, and to the programming unit, to obtain information respectively therefrom regarding the anatomical profile and the pathway for collective use in creating a control input; and
a laser unit for generating a laser beam, and for moving a focal point of the laser beam along the pathway in the vitreous humor in accordance with the control input, to ablate vitreous and floaters located with the optical channel.

11. A system as recited in claim 10 wherein the optical channel is defined relative to an identified axis to extend through the vitreous humor from a distance more than fifty microns (50 μm) from the posterior surface of the lens to a distance more than fifty microns (50 μm) from the retina of the eye.

12. A system as recited in claim 11 wherein the distance r is greater than 2.5 mm.

13. A system as recited in claim 11 wherein the axis is selected from the group consisting of an optical axis, a visual axis, and a central axis.

14. A system as recited in claim 11 wherein the optical channel is located to overlie the macula of the eye.

15. A system as recited in claim 11 wherein the vitreous and floaters are vaporized by the laser unit.

16. A non-transitory, computer-readable medium having executable instructions stored thereon that direct a computer system to perform a process comprising:
identifying an axis for use as a base reference;
establishing an optical channel relative to the axis, wherein the optical channel is defined to extend through an otherwise clear gel-like substance, and wherein the optical channel is substantially cylindrical shaped and extends radially outward to a distance r from the axis; and
ablating the gel-like substance in the optical channel, and deposits suspended therein.

17. A medium as recited in claim 16 wherein the process performed by the executable instructions includes using a laser beam to liquefy the gel-like substance and the deposits in the optical channel by photoalteration.

18. A medium as recited in claim 17 wherein the gel-like substance is vitreous in the vitreous humor of an eye and the optical channel extends between the posterior surface of the lens of the eye, and the retina of the eye, and wherein liquefying the vitreous is accomplished in the optical channel from a distance beyond more than fifty microns (50 μm) from the posterior surface of the lens, and to a distance more than fifty microns (50 μm) in an anterior direction from the retina of the eye.

19. A medium as recited in claim 16 wherein the deposits include at least one floater.

20. A medium as recited in claim 16 wherein the distance r is greater than 2.5 mm.
